(19) United States
(12) Patent Application Publication
(10) Pub. No.: US 2011/0250147 A1
(43) Pub. Date: Oct. 13, 2011

(54) Method and System for Reducing Skin Friction

(75) Inventors: Steven Sher, Beaconsfield (AU); Amit Saha, Beaconsfield (AU)

(73) Assignee: Brands Worldwide Holdings I.P. Pty Limited, Beaconsfield (AU)

(21) Appl. No.: 12/787,399

(22) Filed: May 26, 2010

(30) Foreign Application Priority Data
Apr. 13, 2010 (AU) 2010201452

(51) Int. Cl. A61K 31/78; A61K 9/12

(52) U.S. Cl. 424/43; 424/78.02

(57) ABSTRACT

A film forming material can be used for the preparation of a medicament for pre-treating an area of skin to reduce causes of skin irritation, resulting in a educed risk of acquiring skin chafing and/or blisters. A method of pre-treating can comprise the steps of: providing a dispensing device containing a film forming material; and applying a film forming material to an area of skin, for pre-treating an area of skin to reduce friction and thereby reduce the risk of acquiring chafing and/or a blister.

### FIELD OF THE INVENTION

The present invention relates to skin treatments and in particular to skin treatments for reducing skin irritation resulting from friction against an area of skin.

The invention has been developed primarily for use as a skin pre-treatment for reducing risk of acquiring skin irritation resulting from friction against an area of skin and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

A common cause of skin irritation, resulting in skin chafing and/or blisters, includes friction. Known devices for treatment of skin against irritation resulting from friction or pressure are in the form of dressings (such as patches and bandages), which are typically applied after irritation has occurred. Dressings can be adapted to slip easily over adjacent materials.

### OBJECT OF THE INVENTION

It is an object of the present invention to overcome or ameliorate at least one of the disadvantages of the prior art, or to provide a useful alternative.

It is an object of the invention in its preferred form to provide a skin pre-treatment for reducing risk of acquiring chafing and/or a blister.

### SUMMARY OF THE INVENTION

According to an aspect of the invention there is provided a method of pre-treating an area of skin to reduce the risk of acquiring skin irritation, the method comprising the steps of:
providing a dispensing device containing a film forming material; and
applying film forming material to an area of skin, for pre-treating an area of skin to reduce friction and thereby reduce the risk of acquiring skin irritation that can result from friction against the area of skin.

Preferably, the method reduces the risk of acquiring skin chafing. More preferably, the method reduces the risk of acquiring a blister. According to an aspect of the invention there is provided a use of a film forming material for the preparation of a medicament for pre-treating an area of skin to reduce the risk of acquiring skin irritation that can result from friction against the area of skin. Preferably, the use reduces the risk of acquiring skin chafing. More preferably, the use reduces the risk of acquiring a blister.

Preferably, the film forming material is dissolved in a solvent. More preferably, a dispensing device containing the film forming material is pressurized such that the film forming material can be spray applied in an aerosol format.

Preferably, the film forming material includes an acrylic co-polymer. More preferably, the film forming material includes: dimethylether, acrylic co-polymer, menthol, n-pentane, and ethyl acetate. Most preferably, the film forming material includes:
dimethylether at about 50 percent w/w;
acrylic co-polymer at about 0 5 percent w/w;
menthol at about 0.050 percent w/w;
n-pentane at about 9.450 percent w/w; and
ethyl acetate at about 35.500 percent w/w.

According to an aspect of the invention there is provided a use of a film forming material for the preparation of a medicament for pre-treating an area of skin to reduce the risk of acquiring chafing and/or a blister, as herein described.

### BRIEF DESCRIPTION OF THE DRAWINGS

A preferred embodiment of the invention will now be described, by way of example only, with reference to the accompanying drawings in which:

FIG. 1 is a schematic view of a dispensing device containing a film forming material according to the invention; and

FIG. 2 is a flowchart for a method of applying film forming material according to the invention.

### PREFERRED EMBODIMENT OF THE INVENTION

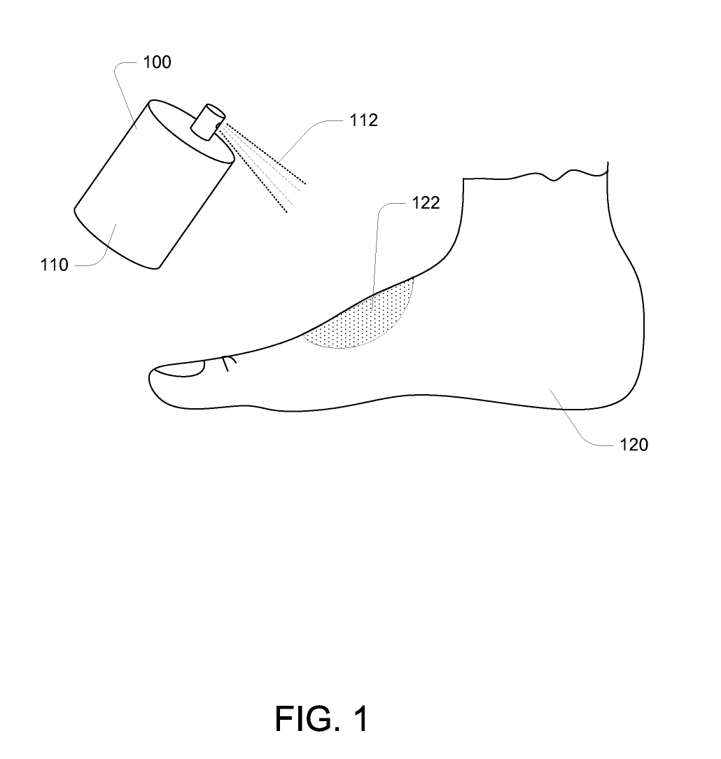

Referring to FIG. 1, a dispensing device 100 contains a film forming material 110. By way of example, the film forming material 110 is in the form of an acrylic co-polymer dissolved in solvent, such as pentane, and pressurized for use in an aerosol form.

Using the aerosol dispensing device 100 to pre-treat an area of skin on a foot 120, a film forming material 110 can be sprayed 112 onto an area of skin 122 for forming a membrane. Preferably, the membrane is water resistant. More preferably, the membrane is air permeable. It will be appreciated that the aerosol dispensing device 100 can pre-treat almost any area of the body to reduce the risk of acquiring chafing and/or a blister.

It will be appreciated that, by way of example only, pre-treatment can cause the formation of a water-resistant air-permeable membrane over an area of skin to provide friction protection and thereby reduce the risk of acquiring chafing and/or a blister.

In an embodiment, the film forming material is in the form of an acrylic co-polymer dissolved in a pentane solvent and pressurized in an aerosol format. By way of example, components of the film forming material can include:

| Components | Percentage | |
|---|---|---|
| Dimethylether | 50% | w/w |
| Acrylic co-polymer | 5% | w/w |
| Menthol | 0.050% | w/w |
| n-Pentane | 9.450% | w/w |
| Ethyl Acetate | 35.500% | w/w |

Similarly, components of the film forming material can include: acrylic acid, dimethylpabamidopropyl laurdimonium tosylate, menthol, and pentane.

It will be appreciated that other film forming materials can be used in accordance with the teaching herein, for pre-treating an area of skin to reduce friction and thereby reduce the risk of acquiring chafing and/or a blister.

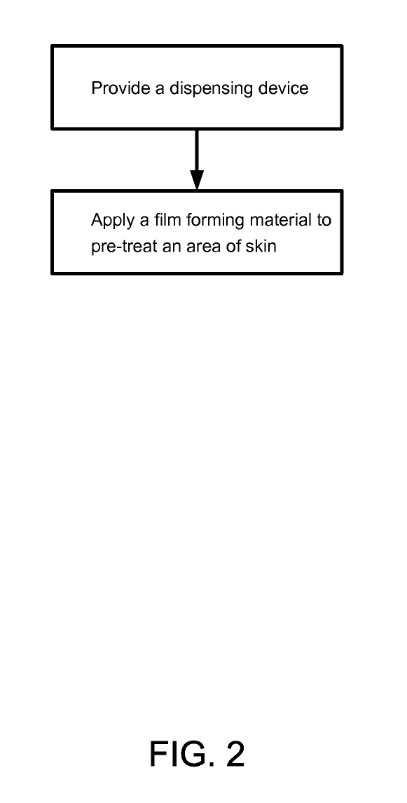

Referring to FIG. 2, an example method 200 of applying a film forming material, includes:
STEP 210: providing a dispensing device containing a film forming material; and
STEP 220: applying a film forming material to an area of skin, for pre-treating an area of skin to reduce friction and thereby reduce the risk of acquiring chafing and/or a blister.

In an embodiment, applying a film forming material comprises spraying a solvent based film forming material onto an area of intact skin for reducing skin friction, which is a common cause of chafing and blisters. It will be appreciated that the method 200 can be both effective and convenient in reducing skin friction.

It will be appreciated that the illustrated method provides a skin pre-treatment for reducing risk of acquiring skin irritation that can result from friction against the area of skin. Preferably, the method reduces the risk of acquiring skin chafing. More preferably, the method reduces the risk of acquiring a blister.

Although the invention has been described with reference to specific examples, it will be appreciated by those skilled in the art that the invention may be embodied in many other forms.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Similarly it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A method of pre-treating an area of skin to reduce the risk of acquiring skin irritation, the method comprising the steps of:
providing a dispensing device containing a film forming material; and
applying a film forming material to an area of skin, for pre-treating an area of skin to reduce friction and thereby reduce the risk of acquiring skin irritation that can result from friction against the area of skin.

2. The method according to claim 1, wherein the film forming material includes an acrylic co-polymer.

3. The method according to claim 1, wherein the film forming material includes:
dimethylether, acrylic co-polymer, menthol, n-pentane, and ethyl acetate.

4. The method according to claim 1, wherein the film forming material includes:
dimethylether at about 50 percent w/w;
acrylic co-polymer at about 5 percent w/w;
menthol at about 0.050 percent w/w;
n-pentane at about 9.450 percent w/w; and
ethyl acetate at about 35.500 percent w/w.

5. The method according to claim 1, wherein the film forming material is dissolved in a solvent.

6. The method according to claim 5, wherein the dispensing device is pressurized such that the film forming material can be spray applied in an aerosol format.

7. The method according to claim 1, wherein the film forming material applied to the area of skin reduces friction to thereby reduce risk of acquiring skin chafing to that area of skin.

8. The method according claim 1, wherein the film forming material applied to the area of skin reduces friction to thereby reduce risk of acquiring a skin blister to that area of skin.

9. A use of a film forming material for the preparation of a medicament for pre-treating an area of skin to reduce the risk of acquiring skin irritation.

10. The use of a film forming material according to claim 9, wherein the film forming material includes an acrylic co-polymer.

11. The use of a film forming material according to claim 9, wherein the film forming material is dissolved in a solvent.

12. The use of a film forming material according to claim 11, wherein a dispensing device is pressurized such that the film forming material can be spray applied in an aerosol format.

13. The use of a film forming material according to claim 9, wherein the film forming material includes:
dimethylether, acrylic co-polymer, menthol, n-pentane, and ethyl acetate.

14. The use of a film forming material according to claim 9, wherein the film forming material includes:
dimethylether at about 50 percent w/w;
acrylic co-polymer at about 0 5 percent w/w;
menthol at about 0.050 percent w/w;
n-pentane at about 9.450 percent w/w; and
ethyl acetate at about 35.500 percent w/w.

15. The use of a film forming material according to claim 9, wherein the film forming material applied to the area of skin reduces friction to thereby reduce risk of acquiring skin chafing to that area of skin.

16. The use of a film forming material according to claim 9, wherein the film forming material applied to the area of skin reduces friction to thereby reduce risk of acquiring a skin blister to that area of skin.
